(19) United States
(12) Patent Application Publication
(10) Pub. No.: US 2011/0229969 A1
(43) Pub. Date: Sep. 22, 2011

(54) Cell Line for Propagation of Highly Attenuated AlphaViruses

(76) Inventors: Volker Sandig, Berlin (DE); Ingo Jordan, Berlin (DE)

(21) Appl. No.: 12/996,549

(22) PCT Filed: Jun. 25, 2009
(86) PCT No.: PCT/EP09/04591

§ 371 Date: Feb. 8, 2011

Related U.S. Application Data
(60) Provisional application No. 61/133,024, filed on Jun. 25, 2008.

(51) Int. Cl. C12N 15/85; C12N 5/10; C12N 7/00

(52) U.S. Cl. 435/455; 435/349; 435/235.1

(57) ABSTRACT

The present invention provides an avian cell that is derived from an avian host cell and stably carries at least one DNA sequence in the cell nucleus encoding an alphavirus polypeptide, a method for preparing such an avian cell, and its use in preparing an alphavirus replican particle.

The present invention provides an avian cell that is derived from an avian host cell and stably carries at least one DNA sequence in the cell nucleus encoding an alphavirus polypeptide, a method for preparing such an avian cell, and its use for preparing an alphavirus replicon particle.

### Introduction

Alphavirus is the arboviral genus of the Togaviridae (the other genus is Rubivirus with a single member that is confined to the human host and transmitted by aerosol). There are more than 40 members in the alphaviruses and two lineages, Old World and New World viruses. The potential for severe epidemics and painful disease is inherent in both lineages. However, symptoms differ: in human patients, infection with members of the former group usually is associated with self-limiting fever and rheumatic manifestations and infection with members of the latter group is associated with encephalitis that sometimes result in permanent sequelae. Typical New World Alphaviruses are Eastern (EEE) and Venezuelan (VEE) equine encephalitis; typical Old World viruses are Semliki Forest (SFV) and Sindbis Viruses (SIN).

Alphaviruses are transmitted by a wide spectrum of different species of mosquitoes to their vertebrate hosts, transmission by tick or other hematophagous arthropodes are exceptions. Typically, either mammalian or avian hosts serve as reservoir for these viruses. The reservoir species maintains sufficiently high titers of virus in the blood stream for the vector to become infected to further disseminate virus. Accidental vertebrate hosts often display the complete spectrum of disease symptoms but usually do not transmit virus to a vector so that the arboviral cycle is disrupted. Reservoir host preference does not segregate with Old/New World lineage. For the Old World viruses, the main vertebrate reservoir are birds for Sindbis and Semliki Forest viruses, mammals (often primates) for O′nyong′nyong and Chikungunya viruses, marsupials for Ross River virus, and humans for Barmah Forest virus. For the New World viruses, the main vertebrate reservoir are mammals (primates and humans) for Mayaro virus, birds for Eastern equine encephalitis virus, and mammals (rodents and horses) for Venezuelan equine encephalitis virus. Western equine encephalitis virus (WEEV) is unusual with reservoirs in both mammals and avians. Indeed, WEEV arises from a natural recombination between viruses related to New World eastern equine encephalitis virus and Old World Sindbis virus (Hahn et al. 1988 in Proc. Natl. Acad. Sci. U.S.A. 85, 5997-6001). The recombination event has occurred within the gene for the structural proteins. Hence, although by serology an Old World virus the disease associated with WEEV infection is inflammation of the brain, typical for New World alphaviruses.

There are also reports of alphaviruses with marine mammals as reservoir and a parasitic louse as vector (Linn et al. 2001 in 3. Virol. 75, 4103-4109) as well as alphaviruses infecting fish such as sleeping disease virus of rainbow trout (Villoing et al. 2000 in 3. Virol. 74, 173-83).

Alphaviruses enter the host cell by receptor mediated endocytosis. They are Baltimore group IV viruses, enveloped virions that encapsidate one single-stranded infectious RNA molecule of positive polarity. Uncoating and release of genomic RNA is triggered by low pH as viral and endosomal membranes fuse. The genomic RNA carries two open reading frames (ORFs), each encoding a single large polyprotein. The first ORF is translated directly off the genomic RNA and processed into non-structural proteins responsible for replication of RNA. Terminal promoters are recognized for production of full length copies where genomic RNA serves as template for negative strand copies and these serve as template for production of genomic RNA.

Replication is extremely rapid and efficient. Within several hours the cell is flushed with viral RNA sequestering the majority of the cellular ribosomes. A molecular switch converts the replication machinery into a transcription complex that recognizes a subgenomic promoter in the central region of the negative-strand copies of the genomic RNA. Transcription from the subgenomic promoter yields mRNA for a polyprotein that is processed into capsid protein and surface glycoproteins to allow generation and release of virus progeny. All steps of viral replication and morphogenesis occur in the cytoplasm.

Alphaviruses are used as efficient expression systems for foreign genes and are in consideration as vaccine vectors. The recombinant payload sometimes is included as a distinct expression cassette with an additional subgenomic promoter. However, these viruses are replication competent with all inherent safety concerns. In more advanced alphavirus expression systems the second ORF is replaced with the gene of interest; the structural proteins are provided by a defective helper virus or by stable inserts in the host cell. Such expression systems are called replicons as viral RNA is amplified in consecutive cycles to express the gene of interest at very high levels in naïve cells or the recipient organism. Because the gene for the structural protein is deleted progeny virus cannot be produced in theory.

However, alphaviruses readily recombine in nature and in artificial systems. This ability poses a significant risk in application as therapeutic vector and has been addressed by two synergisitic techniques, attenuation (weakening in pathogenic potential) and split-helper system (to interfere with recombination). Both approaches are essential for clinical application of alphaviruses and the disadvantages associated with attenuation and split-helper systems must be compensated to obtain an industrially viable therapy or vaccine. To better appreciate the solution provided by the present application the involved mechanism are discussed in greater detail:

Alphaviruses cycle between vertebrate hosts and insect vectors and achieve the required broad host specificity by a combination of molecular properties. They use evolutionary highly conserved host factors to support the infectious cycle. For example, the laminin binding protein is receptor in mosquito and mammalian cells (intriguingly not in avian cells) (Wang et al. 1992 in J. Virol. 66, 4992-5001), and components of the innate immune system are usurped to prevent superinfection and to switch from minus-strand synthesis (replication) to plus-strand synthesis (transcription and morphogenesis) (Sawicki et al. 2003 in J. Virol. 77, 1801-1811). A combination of properties may facilitate attenuation for a certain cell type while maintaining good production properties in another cell type. Due to the layered flexibility of alphavirus host specificity, however, stable and successful attenuation at organism level is difficult to gauge.

Attenuation has been achieved by mutations in untranslated regions as well as in genes either for structural or nonstructural proteins. For example, isolated amino acid changes in the non-structural protein nsP4 of SIN yield a phenotype restricted in replication at temperatures above 34.5° C. (Lemm et al. 1990 in J. Virol. 64, 3001-3011). Surprisingly, multiplication is normal in primary chicken embryo fibroblasts at the permissive temperature but remains restricted in mosquito cells. Decreased virulence for SIN was also achieved by mutations in the surface glycoprotein that lower neurotropism without affecting replication in non-neuronal cells (Tucker and Griffin 1991 in J. Virol. 65, 1551-1557). Certain mutations in VEE glycoprotein did not affect replication in Baby Hamster Kidney (BHK) cells but interfered with spread in C6/36 mosquito cells (Davis et al. 1995 in Virology 212, 102-110). Viable escape mutants emerged in BHK-infected cells and had to be controlled with additional mutations to obtain a more stable attenuation in mice. It is known in the art that especially life vaccines (and alphavirus vectors are useful mainly as minimally purified life vectors) depend in many significant properties on the cell substrate that is used for production. The cell substrate in turn determines the kind of attenuation available for a given vector. As evolution of RNA viruses is rapid isolated attenuating mutations do not pose a significant hurdle against escape mutants. Indeed, the TC-83 vaccine strain of VEE has measurable residual virulence. It is the only VEE strain available for human vaccinées and alternatives are urgently needed (Paessler et al. 2006 in 3. Virol. 80, 2784-2796). Novel attenuation approaches rely on chimeric viruses such as SIN/VEE, where structural proteins are derived from SIN and the replication machinery and cis-active sequence elements come from VEE, or vice versa. Such hybrid viruses were shown to be highly attenuated in mice. The potential for escape to virulence is extremely low as the virus is not equipped with any wild type sequences it could use as template for reversion.

Modern vaccine approaches with alphaviruses (reviewed in Riezebos-Brilman 2006 in 3. Clin. Virol. 35, 233-243) usually rely on suicide vectors that encode the viral polymerase and carry the cognate cis-active elements to allow fulminant RNA replication in the recipient cell but do not encode structural proteins. Such vectors therefore cannot produce progeny particles. For production of the particles the structural proteins are provided by co-infected helper virus, co-transfected helper RNA or stably inserted expression cassettes in a packaging cell line. The RNA encoding the structural protein does not carry packaging signals and thus is not mobilized with the released particles. However, alphaviruses readily recombine probably via a copy-choice mechanism of the viral polymerase (Hajjou et al. 1996 in 3. Virol. 70, 5153-5164). Recombination events can lead to an exchange of the therapeutic gene of interest for the structural gene so that a replication competent virus is created. Attenuation can limit the risk posed to the vacinée if such a replication competent contamination should occur undetected. However, suppression of recombination is the preferred option in clinical application. The probability for recombination has been lowered significantly by the split helper approach where the gene for the structural polyprotein has been separated into two distinct expression cassettes, one encoding the capsid and the other encoding the surface glycoprotein. With this technique two consecutive recombination events must take place, an unlikely coincidence.

To our knowledge, the reliable attenuation of chimeric vectors and the strong interference with recombination in the split helper system come at the price of decreased productivity in BHK or C6/36 mosquito cell lines. We are not aware of a successfull implementation in any helper cell of the highly desirable combination (chimeric virus plus split helper system). Polo et al. (1999 in Proc. Natl. Acad. Sci. USA 96, 4598-4603) describe the split helper system in BHK cells using wild type SIN and SFV components, not attenuated chimeric virus. The reported titers are well below 10̂7 infectious units/ml. Perri et al. (2003 in J. Virol. 77, 10394-10403) describe the chimeric VEE/SIN vector but do not provide a stable packaging cell line—they generate replicon particles by transient expression of helper functions via electroporation of genomic RNA of defective helper viruses. In their investigation of immune stimulation in mice against the gene-of-interest, the VEE replicon (with SIN structural proteins) is preferred to the SIN replicon (with VEE structural proteins). U.S. Pat. No. 6,156,558 also describes transient production of alphavirus replicons by electroporation of alphavirus helper RNA. However, especially for clinical application it is preferred to have a robust system of few independent components (only cell and vector without requirement for additional extraneous nucleic acid for large-scale transient transfection or helper virus for co-infection) and less prone to variation inherent to transient systems. Therefore, to obtain a stable packaging cell the genes for the viral factors must be stably carried by the host cell, either by insertion into the host chromosome or by episomal maintenance. For this genetic manipulation it is important to have an immortal cell line (which precludes primary chicken embryo fibroblasts) that is stable in its karyotype.

Furthermore, the cell line must be highly permissive for the viral vector to be produced in amount and concentration sufficient for clinical application. The cell line should also proliferate in suspension in media free of animal derived components to permit clinically safe production of material.

Additionally, for purposes of vaccine production it is essential that the cell line fulfills regulatory criteria such as complete documentation of cell line derivation and demonstrated absence of adventitious agents. Such a status is not obtainable for BHK cells (commonly used in research on alphaviruses). Also, many insect cell lines including commercial lines such as Tn5 and lines derived from mosquito vectors appear to be plagued by contaminations (for example, see Hirumi et al. 1976 in In Vitro 12, 83-97; Cunningham et al. 1975 in J. Gen. Virol. 27, 97-100; Li et al. 2007 in J. Virol. 81, 10890-10896).

But adventitious agents are not always introduced in the actual process of sample isolation and cell line derivation. Endogenous retroviruses as inherent property of the donor species are particular worrisome: they may have a pathogenic and cancerogenic potential, mask contamination with exogenous retroviruses, by insertion of the provirus may induce changes in expression patterns of the cell, cause mobilization of DNA elements embedded in the genome (Zeilfelder et al. 2007 in Gene 390, 175-9) and integration of sequences from the producer cell or the viral vector into the vaccinée. With respect to mobilization or insertational mutagenesis of cellular genes alphaviruses as RNA vectors that do not convert into DNA provide a very high savety level that should not be invalidated by the producer cell line. Live vaccines and vectors by definition are minimally purified so that a retrovirus contamination will be carried into the final product. For example, reverse transcriptase activity has been detected in live attenuated measles, mumps and yellow fever vaccines produced on cells that harbor endogenous retroviruses (Weissmahr et al. 1997 in J. Virol. 71, 3005-12; Hussain et al. 2003 in J. Virol. 77, 1105-11). Thus, the cell line intended for alphavirus production should not release particle-associated reverse transcriptase. However, especially rodent cells (including BHK; but also the Sf-9 insect cell line) have been shown to harbor high endogenous retroviral activity (for example, Lovatt et al. 1999 in 3. Virol. Methods 82, 185-200).

In summary, the substrate cell line for production of vaccines often is integral to the qualtity and efficacy of the vaccine. The cell line is essential for maintainance of attenuation and may confer certain immunogenic or epigenetic properties such as selected glycosylation or unique proteolytic processing and co-packaging of desired host factors. The Draft Guidance for Industry on “Characterization and Qualification of Cell Substrates and Other Biological Starting Materials Used in the Production of Viral Vaccines for the Prevention and Treatment of Infectious Diseases” by the FDA, CBER, from September, 2006, summarizes well the urgency and importance attached to cell substrates for vaccine production: “The number of different cell substrates used in currently licensed vaccines is limited. The emergence of new infectious diseases necessitates the need for development of new vaccines for agents such as human immunodeficiency virus (HIV), pandemic influenza virus strains, severe acute respiratory syndrome (SARS) virus, and agents of bioterrorism. [ . . . ] Development of new-generation vaccines likely will involve a wider variety of cell substrates, both for the isolation of virus seeds and vectors, and for vaccine manufacture. Selection of a cell substrate influences the safety and purity of the biological product manufactured in it.” This patent application addresses the need and—to our knowledge—for the first time provides a clinically viable solution for production of extremely sophisticated alphavirus vectors.

### SHORT DESCRIPTION OF THE INVENTION

Surprisingly it was found that avian cell lines modified to stably contain alphavirus structural proteins are suitable host cells for the production of highly attenuated alphavirus particles. More specifically, it was found that VEE/SIN chimeric vectors are produced in such cell line at very high titers. Unexpectedly, avian cell lines are highly susceptible and fully permissive to infection with particles that are packaged into SIN envelopes but equipped with VEE replication machinery (non-structural proteins) although the VEE reservoir are mammals, not birds. The split helper system has been incorporated into the cell line and unexpectedly high titers of chimeric virus have been obtained and are herewith demonstrated.

Thus, an avian cell line, termed CR (full designation AGE1.CR), suitable for production of alphavirus vaccine strains and alphaviral vectors for vaccine purposes in human medicine is provided. This avian cell line has been examined for suitability in clinical application and now has also been stably modified to express either one or two species of RNA molecules that allow amplification of messengers for capsid and surface glycoproteins. Thus the invention provides

(1) an avian cell that stably carries at least one DNA sequence encoding an alphavirus polypeptide in the cell nucleus;
(2) a preferred embodiment of (1) above, wherein the avian cell is infected with an alphavirus replicon, wherein
(a) the replicon encodes the viral replication machinery and the avian cell is stably transfected/transformed with at least one structural gene, or
(b) the replicon encodes at least one structural protein and the avian cell expresses the viral replication machinery;
(3) a preferred embodiment of (2) above; wherein the structural gene is separated into its surface and capsid components;
(4) a preferred embodiment of (1) above, wherein the host cell is derived from a permanent cell line with a stable karyotype for at least 50 passages, does not release particle associated reverse transcriptase and grows in medium free of animal components;
(5) a preferred embodiment of (1) above, wherein the permanent host cell is an immortalized avian cell, preferably is immortalized duck cell derived from retina, somite or extraembryonic membrane such as the cell line deposited with the DSMZ under accession number DSM ACC2749;
(6) a method for preparing an avian cell as defined in (1) to (5) above, which comprises stably integrating at least one DNA sequence for the alphavirus polypeptide into the cell nucleus of the host cell as defined in (1) to (5) above;
(7) an alphavirus replicon particle produced by the avian cell (1) to (5) above, which encodes at least one alphavirus structural protein; and
(8) a method to produce alphavirus replicon particles as defined in (1) to (5) above, which comprises contacting a cell of (1) to (5) above with corresponding alphavirus replicon RNA, transducing a cell of (1) to (5) above with alphavirus replicon particles, or transfecting a cell of (1) to (5) above with a nucleic acid sequence encoding at least one alphavirus polypeptide of the replicon.

### SHORT DESCRIPTION OF THE FIGURES

FIG. 1: Schematic of plasmid transfected into CR cells to generate pool C for production alphavirus replicons.

FIG. 2: Fluorescence and phase contrast images of BHK and CR cells transduced with GFP-expressing replicon particles at increasing MOIs, and FACS histogram of GFP signal strength (abscissa) and number of events (ordinate) for each induced culture. Non-induced cells served as background reference. 40000 events were analyzed for each histogram.

FIG. 3: Percentage of cells positive for GFP as function of input MOI. Data is extracted from the FACS analysis of the above FIG. 1.

FIG. 4: Fluorescence and phase contrast images of CR cells stably transfected for expression of SIN structural proteins (pool C) and parental CR cells after challenge with replicon particles at 0.1 MOI. To assay production of progeny replicon diluted supernatant was transferred to BHK monolayers, shown in lower panel. Note cytopathic effect only in cells of pool C and that no progeny replicon was produced on the parental CR cells.

FIG. 5: Fluorescence and phase contrast images of CR cells stably transfected for expression of SIN structural proteins (pool C) and parental CR cells after challenge with replicon particles at various MOIs. Demonstration of spread of replicon is best visible at low MOIs (0.01 or less) and occurs only in monolayers of pool C cells but not in monolayers of parental CR cells. Replicon spreads to neighboring cells within 24 h and consumes the culture within 48 h.

FIG. 6: Result of titration of replicon released by cells of pool C after challenge with increasing number of input replicon. Absolut number of progeny replicon yield is shown by the triangles, burst numbers are given by the columns.

FIG. 7: Transient co-transfection of expression plasmids for VEE replicon and SIN structural proteins into BHK and CR cells. BHK and CR cells were co-transfected with expression plasmids at indicated ratios and released replicon particles were titrated on BHK 48 h after transfection. Columns indicate number of replicon particles produced per transfected cell. The line is associated with the secondary ordinate and gives transfection efficiency. Compare low efficiency here to very high productivity as given in FIG. 8.

FIG. 8: Highly efficient replicon production can be launched by transfection of expression plasmid for replicon RNA into a cell with stably integrated structural genes. Pool C and CR cells were transfected with a common GFP expression plasmid (left column) or expression plasmid for VEE replicon (right column). FACS analysis of GFP-expression plasmid transfection allows determination of transfection efficiency. Diluted Supernatant (0.1 ml of 2 ml initial culture volume) was transferred to BHK monolayers and thus infected BHK cells were quantified for GFP expression (that is, number of replicons in transfected cell supernatant) after 24 hours. The FACS image with the black histogram in the right column shows analysis of the infected BHK cells.

FIG. 9: Agarose gel electrophoresis of a PCR reaction on 100 ng of genomic DNA in panel A to demonstrate presence of genes for structural proteins in CR and BHK cells intended for packaging of replicons, pool C and BHK Z, respectively. Parental cells are negative for this DNA and serve as control. Panel B demonstrates in parallel RT-PCR reactions expression of RNA for non-structural proteins in all transduced cells, RNA for structural proteins only in packaging cells. Note the high signal levels for all species of RNA in pool C compared to BHK Z. H₂O, water only, non-template control; gDNA, genomic DNA; cDNA, reverse-transcribed copy DNA; env, envelope or surface glycoprotein. The size marker is the “1 kb Ladder” from Invitrogen, now Applied Biosystems, (USA); the here visible sizes are 1000, 850, 650, 500, 400, 300, 200 and 100 bp.

FIG. 10: The cells investigated in FIG. 9 for RNA expression shown at the time of harvest in the upper panel, 48 h post transduction. The cells in 6-well plates and 2 ml of culture medium were induced with an MOI of 0.1 replicon particles per cell. The lower panel shows BHK monolayers (also in 6-well plates and 2 ml of culture volume) 36 h post transduction with 100 μl of supernatant from the cultures in the upper panel.

FIG. 11: Proliferation of CR cells in medium free of animal derived components. Panel A shows temperature, angle (amplitude) and rpm (frequency) of platform rotation in the Wave bioreactor in the upper chart and cell proliferation in the lower chart. Cell culture medium in this experiment is Gene Therapy Medium 3. Panel B demonstrates that CR proliferate in a highly desirable homogenous single-cell suspension in a bioreactor.

FIG. 12: Highly sensitive Q-PERT assay on reverse transcriptase activity in duck cells compared to hamster (clearly positive), chicken (positive) and HEK 293 cells (that are known to be negative and define background noise of the assay). Note that duck cells have signal intensities at background level. Panel B shows consensus PCR against endogenous retroviruses of the ALV and EAV group. The duck cells (but not chicken cells) are negative for these proviruses.

### DETAILED DESCRIPTION OF THE INVENTION

According to the invention the avian host cell is a “permanent” cell or “cell line”. A permanent cell or cell line maintains karyotype, morphology and growth properties for at least 50 passages. A permanent cell or cell line can be isolated from natural tumors, can be obtained by repeated passaging of primary cells (including stem cells), or can be obtained by “immortalisation”. “Immortalisation” according to the present invention relates to a the act of transfection of certain functional DNA sequences conferring the potential for at least 50 passages, preferably unlimited number of passages, i.e. “immortality”, to the respective starting cells. It is preferred that the immortalization leading to the cells of embodiment (1) is effected by transfection or transduction with a combination of viral and/or cellular genes (gene(s)), at least one first gene affecting the function of the retinoblastoma protein by mediating disruption of complexes between retinoblastoma proteins and E2F transcription factors and at least one second gene affecting the p53 protein or a family member thereof (as disclosed in WO 2005/042728 which is herewith incorporated by reference in its entirety).

Preferably, the first gene is a gene coding for E1A proteins from mastadenoviruses (preferably from mastadenoviruses of group C) and has the sequence of SEQ ID NO:1. Preferably, the second gene is a gene coding for the adenovirus E1B 55K protein of all groups as given in SEQ ID NO:2. In one preferred aspect the first and second genes are under the control of separate promoters selected independently from PGK-, CMV-, E1- and tk-promoters.

In a preferred embodiment of aspect (4) the host cell is a permanent cell derived from retina, somite or extraembryonic membrane of chicken, duck, goose, quail or the like, preferably from duck. In a particular preferred embodiment of aspect (4) the cell line is cell line 12A07-A10 derived from immortalization of duck extraembryonal membrane cells; or is cell line 17A02 clone H (in the following, AGE1.CR), or is cell line 17A11 clone D or clone B (AGE1.CR.pIX), both derived from embryonic duck retina; or is cell line 12A05 clone A (AGE1.CS), or is cell line 17A11 clone A (AGE1.CS.pIX), both derived from embryonic duck somite.

The cell line 17A11 clone B was deposited on Dec. 8, 2005 at the DMSZ, Deutsche Sammiung von Mikroorganismen and Zellkulturen GmbH, Mascheroder

Weg 1b, 38124 Braunschweig, Germany as DSM ACC2749. This cell line is derived from embryonic retinal cells of duck (Cairina moschata).

Several times in phylogeny of most (if not all) animals retroviruses have infected and integrated into the germ line. These proviruses have been transmitted and spread vertically from ancestral animal to extant species. Primary cells and cell lines may or may not produce particles from these evolutionary remnants. Especially rodent cell lines such as BHK and CHO, but also insect cell lines such as Sf-9, release large numbers of retroviral reverse transcriptase. Among avian cells, primary chicken embryo fibroblasts have been demonstrated to exhibit significant reverse transcriptase activity (Weissmahr et al. 1997 in J. Virol. 71, 3005-12). Since endogenous retroviruses are ubiqitous (for example, see Herniou et al. 1998 in J. Virol. 72, 5955-66) it would be of significant novelty if a permanent cell or cell line can be provided that is both highly permissive for alphavirus replicon production and is free of retroviral activity. Thus, in an especially preferred aspect of (4), additionally this cell line is free of reverse transcriptase activity. Finally, to minimize an external source for contamination, the permanent cell is cultivated in a medium that is free of animal serum and animal derived components, even more preferably a medium that is chemically defined.

The invention is further explained with reference to the following examples that are, however, not to be construed as limiting the invention.

### EXAMPLES

### Example 1

### Cloning of Dual Expression Plasmid for SIN Capsid and Glycoprotein

Construction and production in transient systems of VEE replicons packaged by SIN structural proteins and of SIN replicons packaged by VEE structural proteins is disclosed in patent application WO 02/099035 A2.

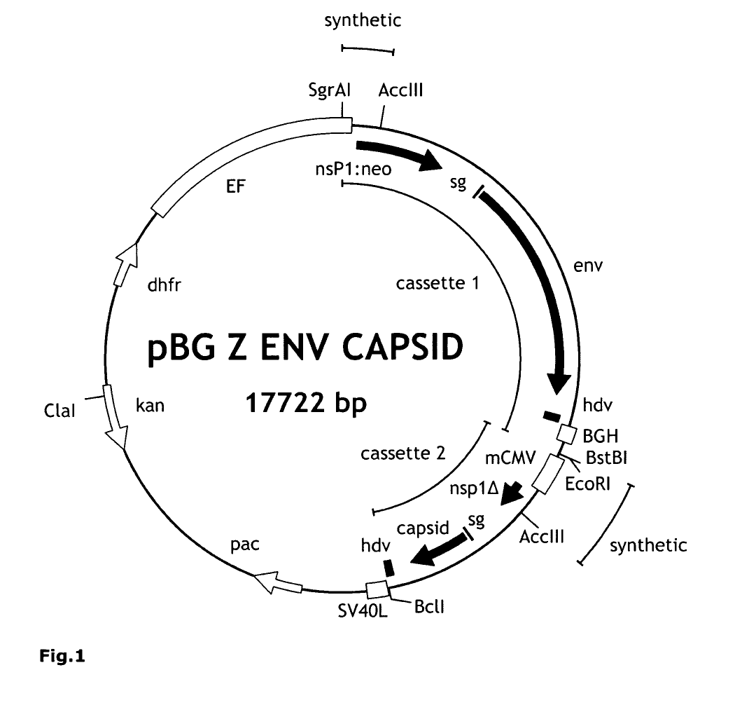

Here, a single expression plasmid, pBG Z ENV CAPSID, was constructed to provide two separate species of VEE-amplifiable RNA encoding the structural proteins of SIN such that glycoprotein and capsid proteins are provided from distinct genes. The general architecture of this plasmid is shown in FIG. 1. The sequences for the structural proteins are identical to the sequences given under GenBank accession number #302363 and are listed here under SEQ ID NO:3 and SEQ ID NO:4.

The cellular polymerase II promoters (human EF2 and mouse CMV promoters as indicated in FIG. 1) and the 5′ untranslated region of VEE (attenuated Trinidad Donkey strain; GenBank #L01442) up to the ATG of the non-structural protein is given as SEQ ID NO:5 and SEQ ID NO:6. The transition from the start site of transcription by polymerase II and first nucleotide of viral untranslated region must be precise and was achieved via a fully synthetic segment that spans the TATA box of the human CMV promoter up to the AccIII sites in the VEE nsP1 gene. The sequence was synthesized on the 5′ side to include unique sites for transfer into EF2 and mouse CMV with SgrAI and EcoRI, respectively. Thus, a novel hybrid promoter is created that carries upstream sequences of mouse CMV or human EF2 promoters up to the TATA box.

The 3′ untranslated region of VEE including artificial poly-adenylation site and hepatitis C virus ribozyme to correctly trim the termini is given in SEQ ID NO:7. In combination, the 5′ and 3′ untranslated regions of VEE allow amplification of the RNA in presence of the VEE non-structural proteins. Expression of the genes for the structural proteins is driven by the VEE subgenomic promoter (denoted as “sg” in FIG. 1), listed as SEQ ID NO:8.

### Example 2

### VEE/SIN Replicons

In the following, GFP replicon refers to amplifiable RNA encoding VEE polymerase and GFP as gene of interest; replicon particle refers to virions that are produced in or released by a cell if GFP replicons and the above described cassettes for SIN structural proteins are co-expressed.

The replicon can be synthesized de-novo by transfection of DNA that encodes its RNA. Such an RNA is disclosed in WO 02/099035 A2 where it is used only in a transient system; the plasmid that launches this RNA by human CMV expression is called A6. The gene for the VEE non-structural protein it expresses is listed as SEQ ID NO:9, including embedded packaging signal for SIN structural proteins. GFP is expressed from the subgenomic promoter, amplification of the RNA is achieved by VEE untranslated termini—all these sequences have been described above.

Transduction is the process of infecting a cell with replicon particles (rather than transfection with replicon expression plasmid). Replicon production in research scale and clinical production scales (that is, in any culture volumes from 100 μl or below to 100 L or beyond) can be initiated either by transduction with particles or transfection with expression plasmid for replicon RNA. Here, transduction with replicon was performed in a minimal volume of cell culture medium (for example, 500 μl of medium for a monolayer of cells in a 3.5 cm diameter dish) for three hours. Thereafter, the infectious suspension was discarded and the cell layer was washed once with medium to remove non-adsorpted replicons. This step is important in any research phase to differentiate in subsequent analysis residual input particles from newly produced particles. To replace the medium is not important in the actual production process. Thereafter, medium was added to normal volumes (for example, 2 ml of medium for a monolayer of cells in a 3.5 cm diameter dish).

The amount of replicon added is determined by a given multiplicity of infection (MOI) which is the number of replicons added per cell in the reaction.

Replicons are quantified directly from the supernatant by dilution of the solution to be titered so that 0.1 to 10% of 1×10⁶ BHK cells are infected. Higher infection rates may underestimate the titer as probability increases that cells are hit by more than one replicon.

After 24 hours, the cells are resuspended by trypsination and precise proportion of GFP-positive to GFP-negative cells is determined by Fluorescence Activated Cell Sorting/Counting (FACS). From this proportion it is possible to determine the titer.

For example, let us assume that infection of the BHK monolayer of 1×10⁶ cells with 20 μl from a 10-fold dilution of particle suspension yields a proportion of 8% in 4×10⁴ FACS events. Proportion times events yields 3200 infected cells. Only 1×10⁴ cells were counted in the FACS out of 1×10⁶ cells in the sample so actual titer in the inoculum is 3200×25 in 20 μl or 3200×25×50=4×10⁶ infectious units/ml. Initial dilution from seed to inoculum was 10-fold so actual titer in the seed is 4×10⁷ infectious units/ml.

### Example 3

### Susceptibility of CR Cells

The structural proteins are derived from a virus adapted to passage cycle between mosquito and birds (SIN). The non-structural proteins forming the replication machinery are derived from a virus with mammals as reservoir host (VEE). To confirm that the AGE1.CR duck cells are good hosts for chimeric alphaviruses a population first was re-adapted to adherent growth from the suspension culture by serial passage in DMEM/F12 medium containing 5% fetal calf serum (FCS). Suspension cells proliferating in medium free of animal derived components are preferred in industrial application but results are more difficult to interpret. Hence, adherent monolayers (rather than suspension cultures) of 1.3×10⁶ CR cells and 1×10⁶ BHK cells as parallel reference were transduced with GFP-expressing replicons at increasing MOI (0.1, 1, 5 and 10).

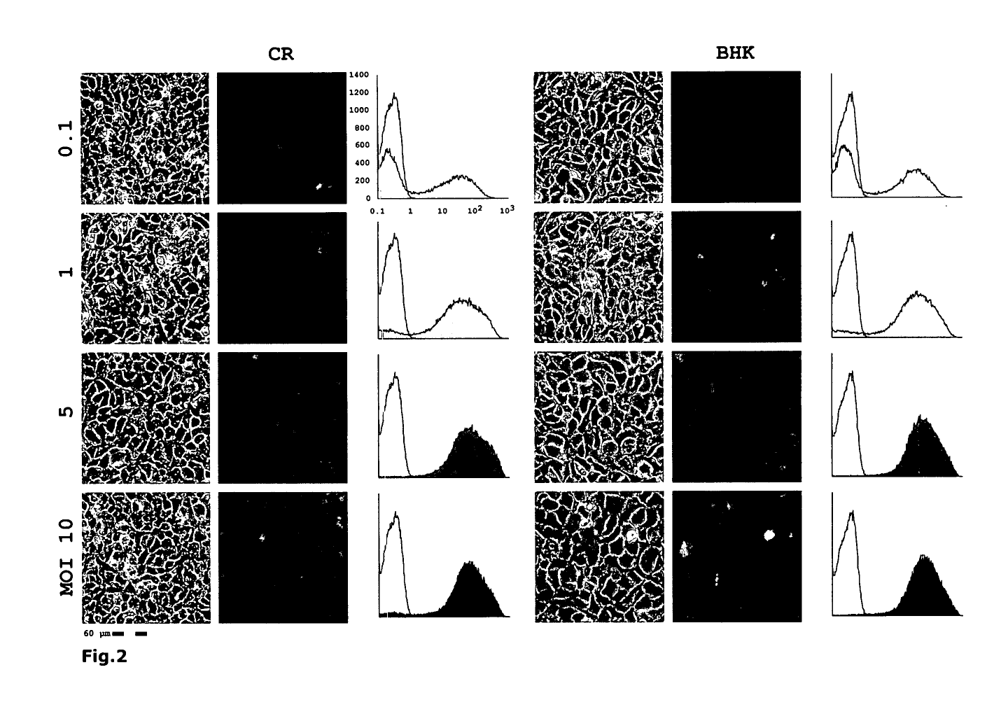

FIG. 2 compares appearance of the cell monolayers of BHK and CR cells at different MOIs. Expression of GFP allows direct visualization of the infection process and quantification by FACS (fluorescence activated cell counting/sorting). The experiment demonstrates that there are no differences in adsorption of virus to CR or BHK, with BHK known to be highly susceptible and considered gold standard in these experiments.

24 hours post transduction, cells were detached by trypsination and quantified by FACS analysis (Partec FloMax device, gain set to 164 units). Two independent experiments were performed, both with highly similar results. FIG. 2 shows the FACS histogram next to the corresponding culture.

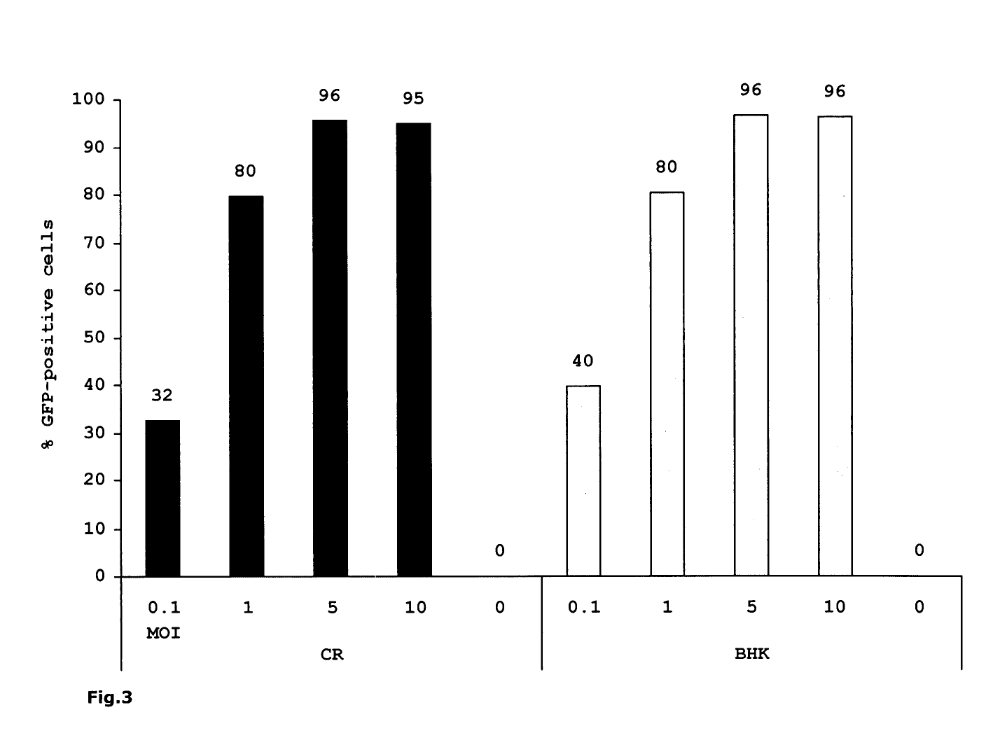

The column chart in FIG. 3 shows the area of the curves for the GFP-positive cells. Susceptibility of CR and BHK clearly is congruent for all MOIs that were tested. In summary, CR cells are highly susceptible to VEE/SIN-hybrid infection. Compared to BHK no differences were observed in GFP signal frequency from replicon particles upon transduction of cell monolayers.

### Example 4

### Results on Induction of Stable Cell Pools

CR cells were transfected with a liposomal preparation (Effectene, Qiagen, Germany, according to the instructions of the manufacturer) of pBG Z ENV CAPSID that has been linearized by digestion with ClaI. After 24 hours, medium was replaced and after further 24 hours geneticin (Gibco, USA) was added to 300 pg/ml to select for cells that stably express the transfected cassette.

At the first passage individual clones were isolated by limiting dilution. The remainder was passaged in presence of geneticin as mixed population that stably express the cassettes for alphaviral structural proteins (termed “pool C” in the following).

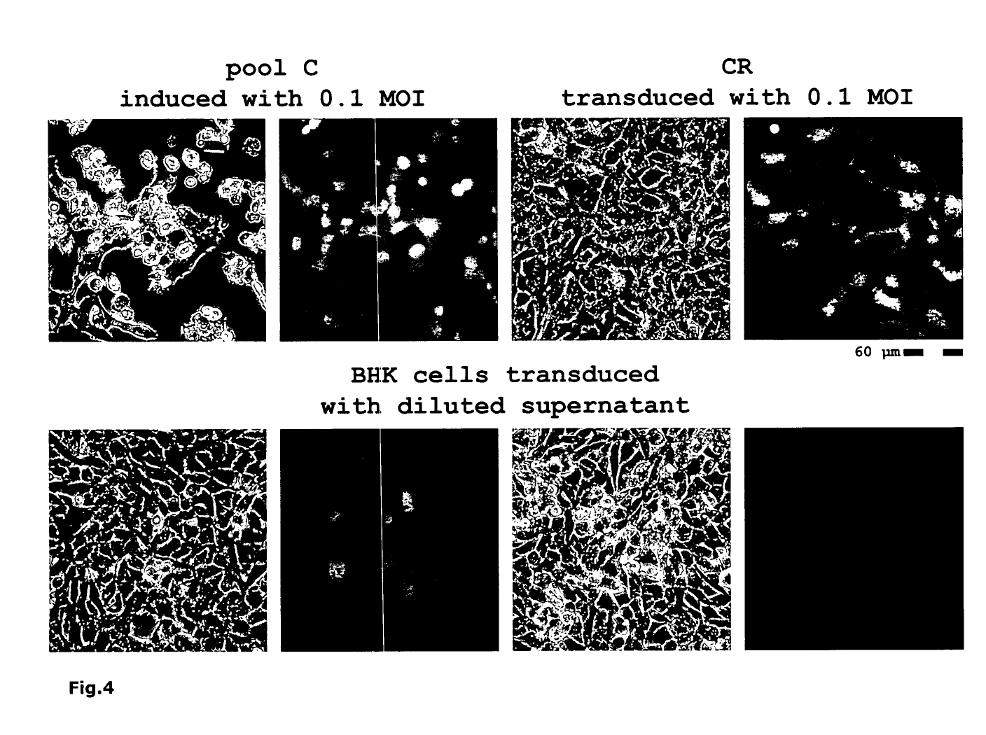

Monolayers of cells from pool C and from the parental CR cell line in 6-well plates were induced with GFP-replicon particles at various MOIs (FIG. 4 upper panel, shown for MOI of 0.1). 48 h after induction, supernatant was diluted 200-fold and transferred to BHK cells; quantification of GFP signal in BHK by FACS was performed 24 h after transfer of supernatant (the lower panel in FIG. 4 shows BHK cells prior to FACS analysis).

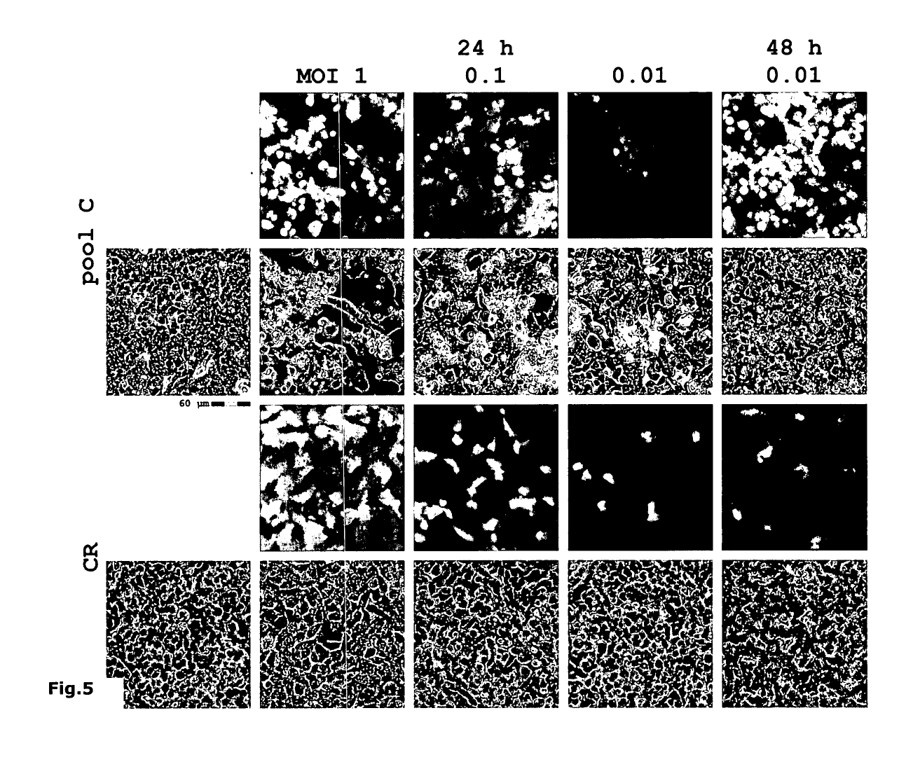

FIG. 5 shows pool C cells and parental CR cells induced with increasing MOIs, from 0.01 to 1, 24 hours post inoculation. Induced pool C cells exhibit CPE from alphavirus replication whereas parental CR cells show strong GFP signal but limited CPE. Expected spread of GFP signal only in pool C due to replication of the SIN/VEE chimera is best visible at low MOIs of 0.01 or less. Compare last two columns where cultures induced with 0.01 MOI are shown 24 and 48 hours post inoculation. There is no spread of GFP in the parental CR cells.

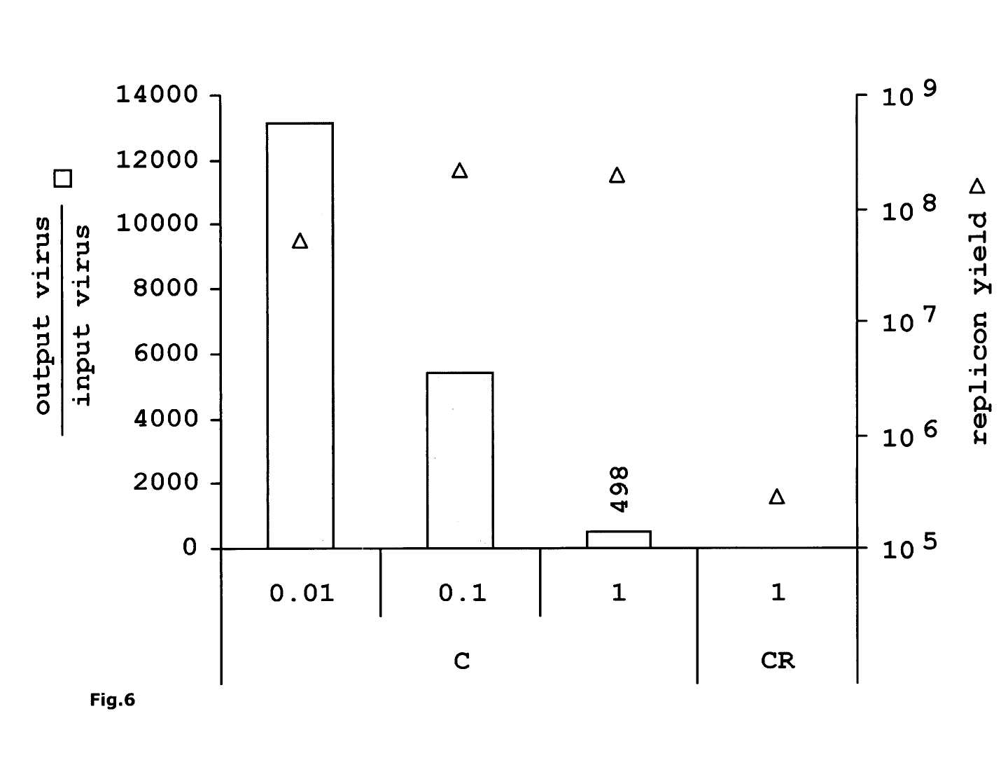

The column chart in FIG. 6 summarizes absolute yields and burst rates obtained for pool C. Input virus carry-over and background noise of the FACS analysis was determined by titration of supernatant from cr cells transduced with replicon. To briefly describe the procedure and derivation of results: 4×10⁵ stably transfected pool C cells or parental CR cells as control where induced with replicons at an MOI of 1 and 0.1. This corresponds to 4×10⁵ or 4×10⁴ input replicons, respectively.

The induced monolayer was washed within 24 h. Culture volume for the next 24 h was 2 mL, and 10 μl thereof (i.e. 1/200 of the total yield) was transferred to 2×10⁶ BHK cells, 48 h after induction. FACS analysis of these BHK cells 24 h post infection with supernatant gave GFP signal in 49.82% for pool C at an MOI of 1, 54.34% at an MOI 0.1, 13.16% at an MOI of 0.01 and 0.07% for the parallel parental CR control. As an example, the calculation for MOI 0.1:
0.54×2×10⁶=1.08×10⁶ output replicons in 10 μl, and
2.16×10⁸ replicon particles in 2 ml, which is final yield.

Burst size is given by the ratio of output virus to input virus. At an MOI of 0.1 this is: 2.16×10⁸/4×10⁴=5400

Burst size at an MOI of 1 roughly corresponds to the productivity of a single cell, here a surprisingly high 498 replicon particles per cell. At an MOI as low as 0.01 and probably even lower replication is launched successfully in the culture although final yields may be lower in this non-optimized system compared to a launch with multiplicities of 0.1 or 1. Results improve with a homogenous clonal culture and process optimization.

### Example 5

### Transfection to Launch Replicon Production

Producer cells can launch replicon release after induction with replicon particles or after transfection of in-vitro transcribed RNA or an expression plasmid for that RNA. This fact is surprising only for transfection of DNA (less so for transfection of replicon RNA as this has already been demonstrated). Transfection of DNA is more convenient as DNA is more stable than RNA. DNA just like RNA can be produced in high amounts at qualities suitable for clinical application. The main difference between transfection of expression plasmid DNA compared to transfection of RNA encoding replicon and amplifiable RNA for structural proteins is the number of molecules likely to be available in the host cell: DNA expression plasmid will produce massive amounts of replicon (or amplifiable) RNA of a given polarity. Considering that in the natural infection only one virus enters a cell and thus only a single copy of genomic RNA is initially available the transfection of expression plasmid DNA may overwhelm the viral replication and transcription machinery. The viral polymerase by a time dependent switch (that ironically may be triggered by the innate immune system of the cell) switches from minus-strand copy generation to transcription from the sub-genomic promoter. At that time in the viral cycle, evolution has adjusted for an optimal ratio of copies of genomic and antigenomic RNA. This ratio most likely is disturbed upon transfection of DNA expression plasmids (especially if the driving promoter is strong such as a CMV promoter) and may depress productivity in transient systems. Furthermore, usually not all cells will be transfected with both plasmids (or three plasmids if the split helper is not unified into a single plasmid). Non-transfected cells may adsorb replicons produced by the co-transfected cells but will not release progeny. Thus, with decreasing transfection efficiency replicon production will be increasingly quenched.

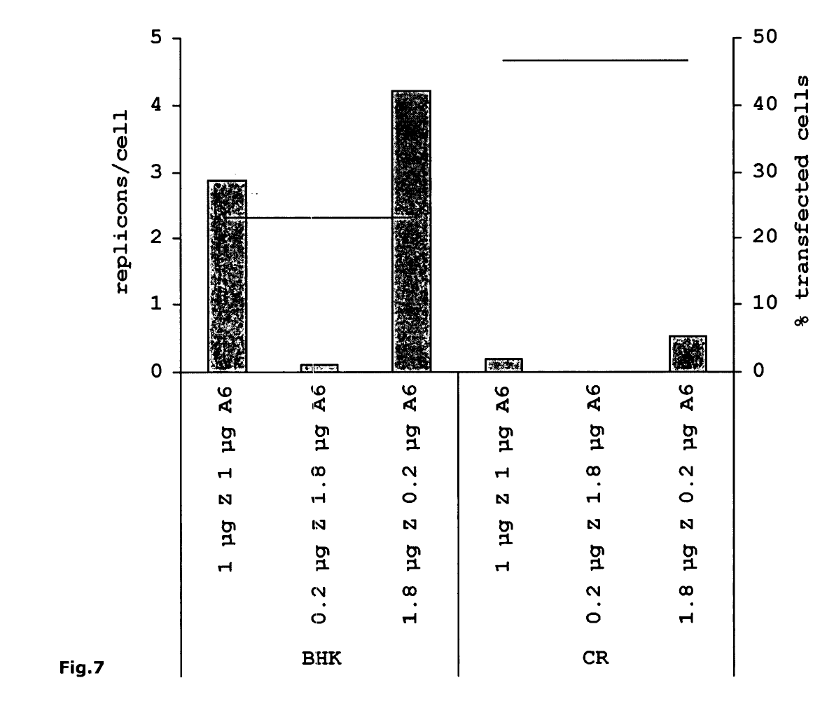

Results from a fully transient approach is shown in FIG. 7: 4×10̂5 CR or BHK cells were transfected with liposomal preparation of pBG Z ENV CAPSID mixed at various ratios with replicon expression plasmid A6. Transfection efficiency was determined by FACS quantification of cells co-transfected with 1 μg A6 together with 1 μg of an inert pUC plasmid and was 23% for BHK and 47% for CR. 48 hours post transfection 100 μl supernatant from each of the transfected cultures was assayed on BHK cells for replicon particle yield. FIG. 7 demonstrates that a plasmid based production system for highly attenuated alphavirus appears to be very difficult to implement, if it is possible at all. Replicon production clearly is sensitive to the amount of A6 in a cotransfection but never reaches levels beyond 5 particles per cell. Surprisingly, productivity is lower in CR than in BHK cells although transfection efficiency (optimized for CR) was significantly higher in the CR line.

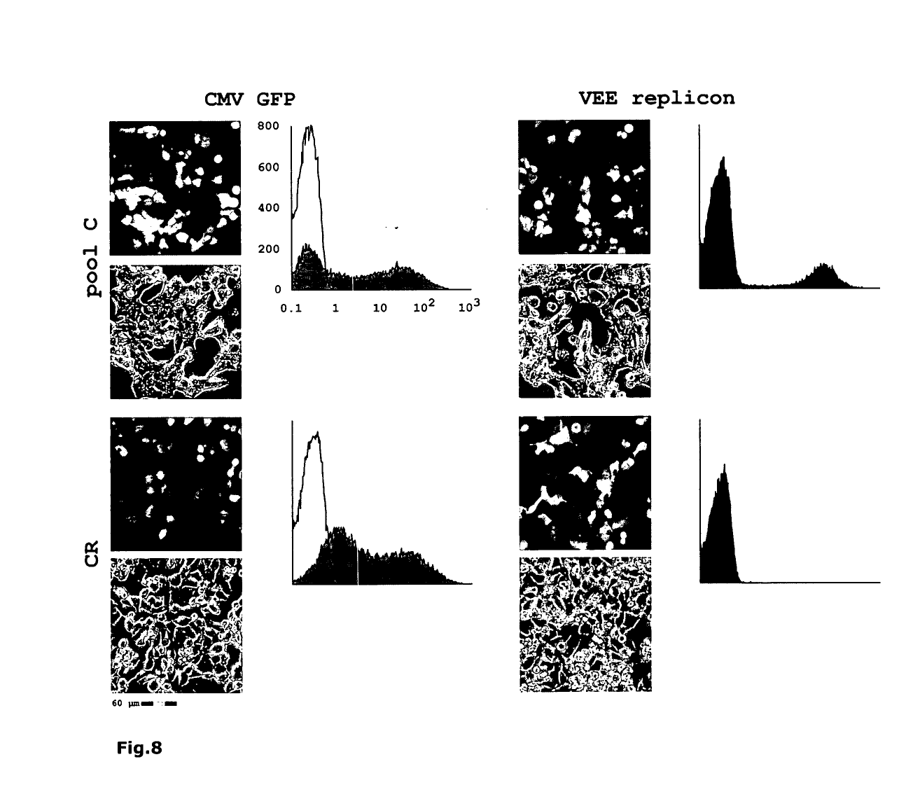

To determine whether launching of a production process by transfection of expression plasmid for replicon RNA is feasible in stable producer cells pool C also was transfected with A6. FIG. 8 demonstrates that pool C initiates robust replicon production upon transfection of VEE replicon expression plasmid A6: in the left panel cells are shown that were transfected with a common GFP expression plasmid to determine transfection efficiency. The FACS histogram gives in the light gray curve non-transfected background and in the dark grey curve cells with GFP signal. The number of events in the positive fraction over total number of events (40 000) constitute 58% for CR and 37% for pool C. Cells transfected with A6 are shown in the right panel, 24 h post transfection. BHK cells were inoculated with 10 μl of supernatant of the permanent duck cells 48 h post transfection. After 24 h, these BHK cells were analysed by FACS with the corresponding histograms shown in the right panel. 4×10̂5 cells were transfected in 2 ml of culture volume, 10 μl was transferred to 2×10̂6 BHK cells for titration. 19% of the BHK cells were positive with GFP from progeny replicon in pool C. Thus, for pool C, a total yield of 7.5×10̂7 particles was determined on BHK; the parental CR did not release any replicons, as expected. From the number of transfected cells (0.58×4×10̂5) a high productivity of 508 replicons/cell was determined.

### Example 5

### Unique Properties of Avian Cell Line

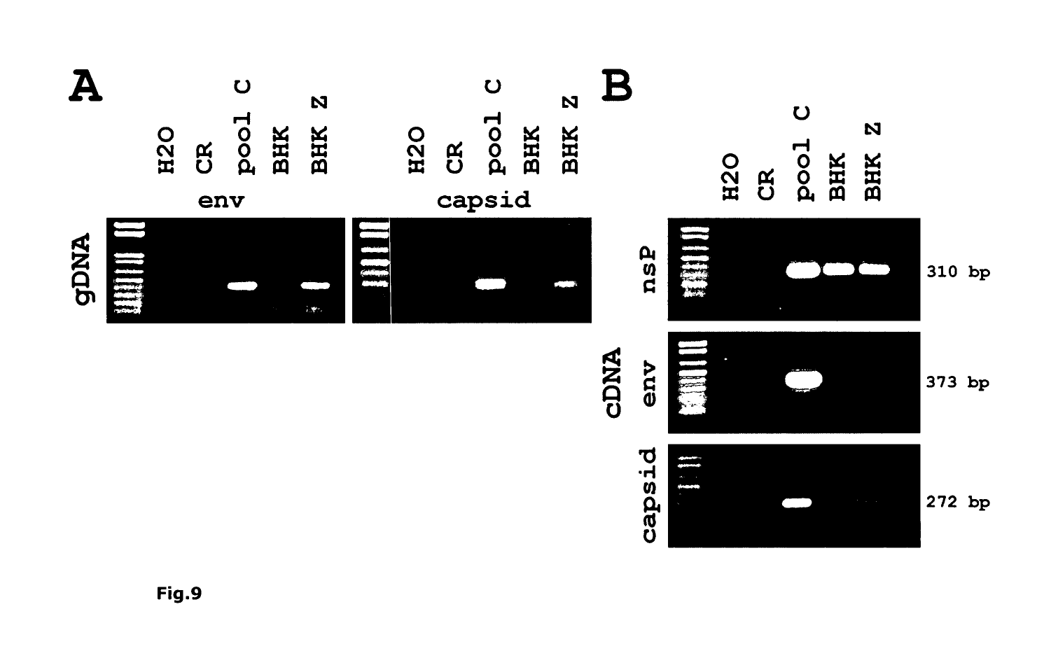

The results obtained with the avian cell line are surprising especially as the chimeric system is expected to be highly active in BHK cells but was found not to produce replicon there even at induction with higher MOI. BHK cells for various reasons are not admissable for production of vaccines and vectors intended for medicinal use; they have spontaneously formed posing unknown risks to recipients of derived biologicals, are highly tumorigenic and their history is inadequately reported. Nevertheless, as a reference for the development in CR cells the chimeric system was also used in BHK cells. All steps from linearization of pBG Z Env Capsid plasmid to transfection and selection were done analogous to the procedure described above for generation of the CR pool C. Stable introduction of pBG Z Env Capsid in BHK was confirmed by PCR on genomic DNA isolated from approx. 2×10⁶ cells with the QIAamp DNA Blood Mini Kit (from Qiagen, Germany). Genomic DNA from parental BHK and CR cells as well as CR pool C cells served as controls. PCR was performed using primers i220 (CAG CGT AAC GGT TAG CAT AG; SEQ ID NO:10) and i221 (CGT TTG GTC GCT CTT ATA GG; SEQ ID NO:11) for detection of surface glycoprotein and primers i222 (CAT TGG ACA GGC AAC TAG AC; SEQ ID NO:12) and i223 (CTT TCA CGT GCA GAG GTT TC; SEQ ID NO:13) for detection of capsid. Reaction conditions are 1 μl spin column eluate in a final volume of 20 μl in presence of 188 μM of all four dNTPs and 1.2 μM of each primer with 0.2 μl of Taq DNA Polymerase from Qiagen (Germany) in the buffer supplied by the manufacturer. The thermocycler was programmed to 2 min at 95° C. followed by 25 cycles of 15 s at 95° C., 20 s at 52° C. and 20 s at 72° C. The amplification products are shown in FIG. 9A, the fragment derived from the surface glycoprotein is 373 by in size, the fragment from the capsid is 272 bp.

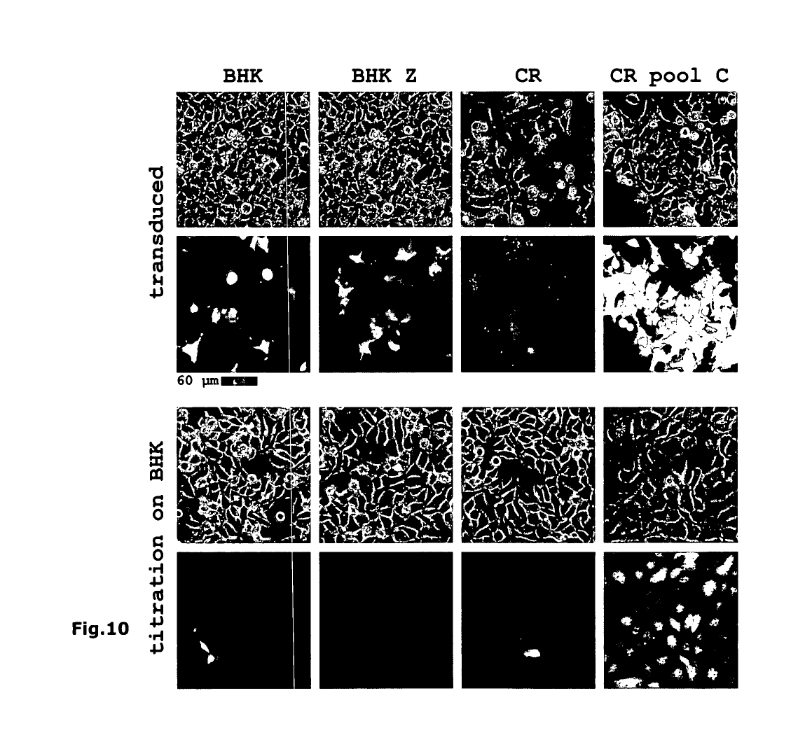

The stably transfected BHK cells were named “BHK Z”. BHK, BHK Z, CR and pool C cells were provided as monolayers and induced as described above. 48 hours after induction with MOI of 0.1, total RNA was isolated using the NucleoSpin RNA II kit (from Macherey Nagel, Germany) according to the instructions of the manufacturer. Total RNA was reverse transcribed with Cloned AMV First-Strand cDNA Synthesis Kit (from Invitrogen, USA) according to the instructions and in the buffers provided by the manufacturer: 500 ng of total RNA was activated with 100 ng of random hexamers by incubation for 10 min at 25° C.; reverse transcription was allowed for 50 min at 50° C. followed by inactivation of enzyme for 5 min at 85° C. 0.25 μl each of the cDNA reaction was subjected to PCR reactions with primers i218 and i219 (CTT CCA TGA ATT CGC CTA CG and TCT GAG AGT ACC TGC ATG AC, respectively, SEQ ID NO:14 and SEQ ID NO:15), i220 and i221, and i222 and i223 for detection of RNA for non-structural proteins, surface glycoprotein, and capsid, respectively (35 cycles for detection of capsid, 25 cycles for all other PCR reactions; conditions as described above with genomic DNA as sample). FIG. 9B shows images of the RT-PCR products in 1% agarose gels stained with ethidium bromide. High levels of RNA for non-structural proteins (encoded by the replicon; there, the amplicon is 310 by in size) and structural proteins (encoded by the split helper embedded into the cellular genome) are evident in CR cells indicating both successful induction and robust amplification. However, in BHK cells only very low levels of RNAs for structural proteins are produced demonstrating failed amplification. RNAs for non-structural RNAs are present indicating that induction was successful, albeit abortive. Consequently, transfer of supernatant from CR pool C cells to a BHK indicator cell monolayer yields high levels of transduction with GFP. GFP signal after transfer of supernatant from the BHK packaging cells is consistent with transfer only of input replicon rather than production of progeny particles. Induced cells and BHK cells transduced with the supernatant are shown in FIG. 10. A surprising property of the described avian cell lines allows generation of stable packaging cells for highly attenuated alphaviruses.

### Example 6

### Proliferation in Media Free of Animal Derived Components

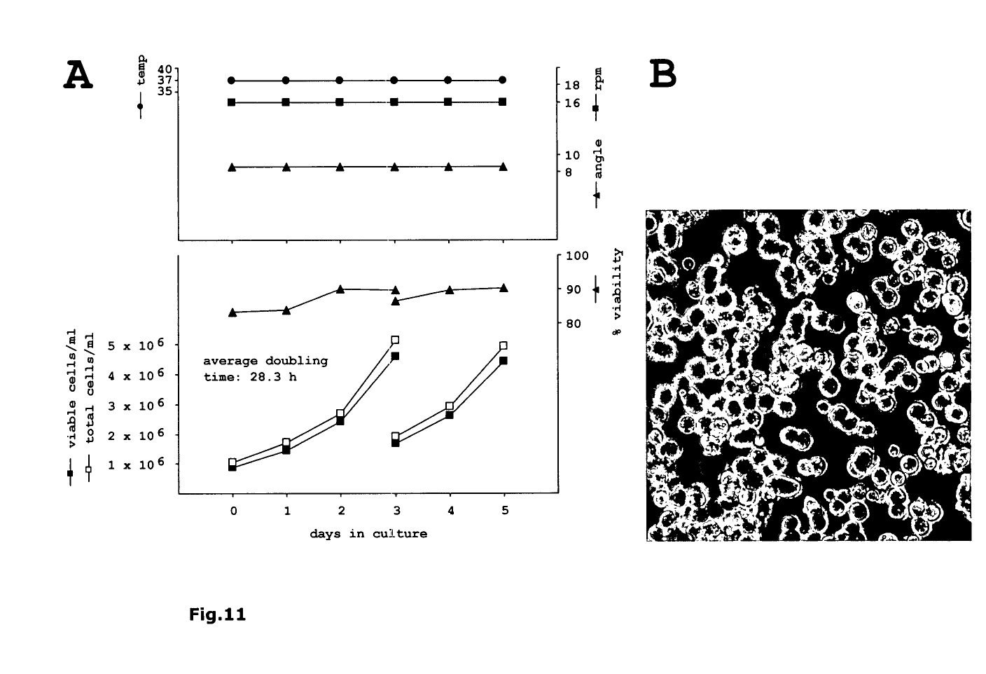

For production of biopharmaceutical material for clinical application it is extremely desirable that the cell line proliferates well in absence of calf serum, even more preferably in media free of animal-derived components and without carrier. The CR cell line has been adapted to growth in a number of freely-available commercial media formulated without animal-derived components such as Adenovirus Expression Medium (Invitrogen #12582-011, USA), Gene Therapy Medium 3 (Sigma #G9916, USA) or ProPer 1 (Lonza #BE02-028Q, Switzerland); all media supplemented to 1× concentration with GlutaMAX I (Gibco #35050). Proliferation of the CR cells is extremely robust in various bioreactors including the Wave bioreactor. The advantage of the Wave bioreactor is that it provides a disposable system especially suitable for production of infectious material. FIG. 11 demonstrates proliferation of the CR cell line in the Wave bioreactor and thus a final requirement for clinical application.

### Example 7

### Assay for Retroviral Activity

The Draft Guidance for Industry on “Characterization and Qualification of Cell Substrates and Other Biological Starting Materials Used in the Production of Viral Vaccines for the Prevention and Treatment of Infectious Diseases” by the FDA, CBER, from September, 2006, clearly advises that “rodent cell lines are presumed to be capable of producing endogenous retroviruses. [ . . . ] You should only use rodent cell lines if your product can be sufficiently purified to demonstrate levels of viral clearance that assure the final product is not contaminated with retroviral particles.” Since endogenous retroviruses are ubiqitous it would be of significant novelty if a cell line can be provided that is both highly permissive for alphavirus replicon production and is free of retroviral activity. Only from such a cell line a clinical product can be obtained that tolerates only minimal purification to maintain activity.

In the above examples high productivity of the permanent CR cells for highly attenuated alphaviruses has been demonstrated. This example via two independent, complementating assays (consensus PCR and highly sensitive Q-PERT) demonstrates that—contrary to rodent cells such as BHK and CHO—the provided avian cells serendipitously are free of retroviral activity.

Retrovirus phylogeny in birds is complex and such a considerable number of endogenous retroviruses have colonized the chicken genome that extensive breeding merely eliminated one of several families of endogenous retroviruses (Boyce-Jacino et al. 1992 in J. Virol 66, 4919-29).

Chicken cells harbor several families of proviruses including the endogenous avian retroviruses (EAV) and endogenous avian leukosis viruses (ALV, also sometimes listed as leukosis/sarcosis group, ALSV). Primers were designed against consensus sequences of various retroviruses of the ALV and EAV groups. Primer V215, TGG TGA CCC CGA CGT GAT; SEQ ID NO:16 is directed against the conserved Trp primer-binding site of ALV and EAV. Reverse primers V216, CCT ATT TCC TTC TTA GAA GGA GA; SEQ ID NO:17 and V217, CGA TCT CCT TCC CGG AAG GAG T; SEQ ID NO:18 are located downstream in gag (the capsid gene of retroviruses). PCR with primers V215/V216 is designed to pick up ALV and with primers V215/V217 is designed to pick up EAV. For example, PCR on avian leukosis virus subgroup C, also named Prague strain of Rous sarcoma virus subgroup C, yields an amplicon of 358 by on sequence #V01197 in the EMBL data bank. For both primer combinations the thermocycler was programmed to 94° C. 3 min; 35 cycles of 94° C. 30 s, 54° C. 30 s, and 72° C. 40 s in a standard PCR reaction with Taq polymerase (Qiagen, Germany) in provided buffers.

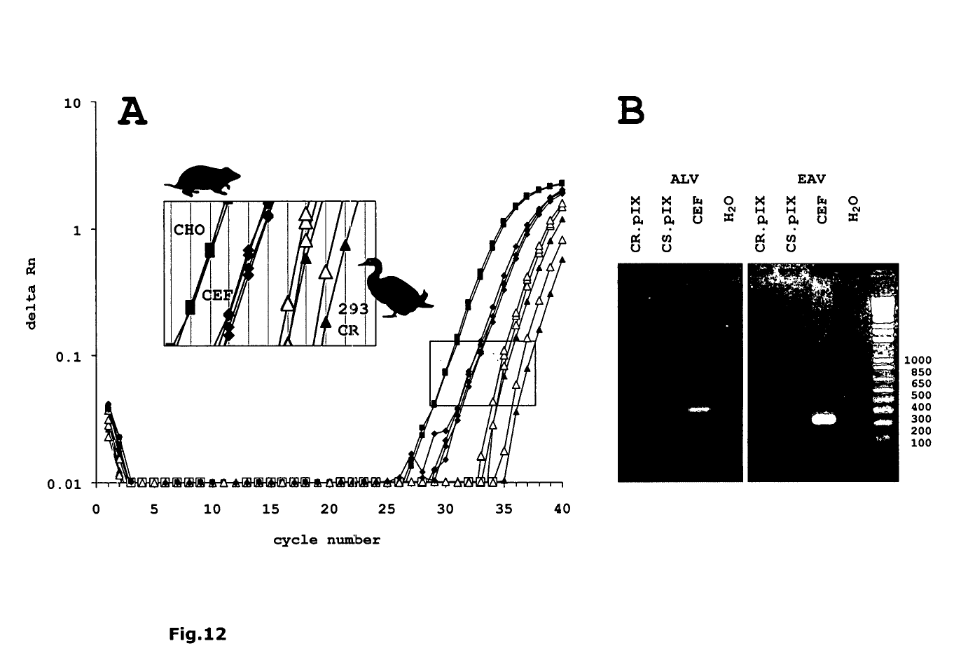

FIG. 12 in panel B shows amplification products for ALV and EAV only in primary chicken cells but not in CR.pIX or CS.pIX cells (and by inference, as proviruses are stably integrated into the genome, not in the parental CR and CS lines).

Q-PERT used here (quantitative probe-based product enhanced PCR for reverse transcriptase) is a modification from the literature (Lovatt et al 1999 in J. Virol. Methods 82, 185-200). This assay uses the reverse transcriptase enzyme of (contaminating) retroviruses to convert a model substrate (such as genomic RNA of brome mosaic virus, BMV, #D1541, from Promega Corp, USA) into a cDNA of known sequence. This cDNA subsequently is demonstrated and quantified by real-time PCR. The specific primer used for reverse transcription is GCC TTT GAG AGT TAC TCT TTG; SEQ ID NO:19; the primers used for cDNA amplification are AAA CAC TGT ACG GCA CCC GCA TT; SEQ ID NO:20 and GCC TTT GAG AGT TAC TCT TTG; SEQ ID NO:21.

To briefly describe the procedure: retroviruses, if present, were enriched from culture supernatant by ultracentrifugation with 100 000×g through a barrier of 20% sucrose in PBS to remove cellular debris. Virions in the invisible pellet were resuspended into lysis buffer (50 mM Tris pH 7.8, 80 mM KCI, 2.5 mM DTT, 0.75 mM EDTA, 0.5% Triton X-100) to release the reverse transcriptase and mixed with substrate buffer (10 mM each of dATP, dCTP, dGTP, and dTTP; 15 μM specific primer; and 0.5 mg/ml fragmented herring sperm DNA [Promega Corp, #D1811]) containing the model RNA (5 μg/ml Brome Mosaic Virus RNA) that is reverse transcribed if RT activity is present in the sample. cDNA from the model RNA is amplified by PCR and detected via SYBR green fluorescence in an AB 7000 Sequence Detection System using the QPCR SYBR Green ROX Mix #AB-1163 from Abgene, UK, according to the instructions of the manufacturer.

FIG. 12 in panel A demonstrates strong RT activity in CHO cells as expected from a rodent cell line. Q-PERT is a very sensitive method and false positives at the limit of detection sensitivity are known in the literature (for example, Fan et al. 2006 in 3. Clin. Virol. 37, 305-12); these signals are usually caused by normal cellular DNA polymerases with RT activity such as polymerase alpha or mitochondrial polymerase gamma. Thus, with CHO cells as positive control and human 293 cells free of retroviral activity as background control a bracket is defined that allows interpretation of unknown RT activity in the supernatant of cell cultures (FIG. 12, bold squares and bold triangles). Moderate RT-activity was observed in chicken embryo fibroblasts (FIG. 12, bold diamonds). The signal for RT activity in the duck cell supernatant (FIG. 12, open triangles) was congruent with the signal for RT activity in 293 cells. Thus, contrary to rodent (and chicken) cells the described duck cells do not exhibit RT activity and fulfill an essential attribute for suitability in pharmaceutical applications.

### SEQUENCE LISTING, FREE TEXT

### SEQ ID 1: E1A CASSETTE

### SEQ ID 2: E1B CASSETTE

### SEQ ID 3: SIN CAPSID

### SEQ ID 4: SIN GLYCOPROTEIN

### SEQ ID 5: EF2 AND VEE UTR UP TO ATG

SEQ ID 6: mCMV AND VEE UTR UP TO ATG

### SEQ ID 7: VEE 3′ UTR AND HDV

### SEQ ID 8: VEE sg

### SEQ ID 9: VEE nsP AND SIN PACKAGING SIGNAL

### SEQ ID 10: PRIMER

### SEQ ID 11: PRIMER

### SEQ ID 12: PRIMER

### SEQ ID 13: PRIMER

### SEQ ID 14: PRIMER

### SEQ ID 15: PRIMER

### SEQ ID 16: PRIMER

### SEQ ID 17: PRIMER

### SEQ ID 18: PRIMER

### SEQ ID 19: PRIMER

### SEQ ID 20: PRIMER

SEQ ID 21: PRIMER

We claim:

1. An avian cell that is derived from a permanent avian host cell and stably carries at least one DNA sequence encoding an alphavirus polypeptide in the cell nucleus.

2. The avian cell of claim 1, wherein the avian host cell is infected with an alphavirus replicon, wherein
(a) the replicon encodes at least one non-structural protein and the avian host cell is stably transfected/transformed with at least one structural gene, or
(b) the replicon encodes at least one structural protein and the avian host cell is stably transfected/transformed with a gene for at least one non-structural protein.

3. The avian cell of claim 2, wherein
(a) the structural protein is derived from an alphavirus with non-mammalian animals as reservoir host and the non-structural protein is derived from an alphavirus with mammals as reservoir host species, or
(b) the structural protein is derived from an alphavirus with mammals as reservoir host and the non-structural protein is derived from an alphavirus with non-mammalian animals as reservoir host species.

4. The avian cell of claim 2 wherein
(i) the structural gene, including the capsid and surface glycoprotein, is separated into at least two units; and/or
(ii) the structural gene and the non-structural gene, including genes nsP1 through 4, are derived from different alphaviruses; and/or
(iii) the alphavirus is selected from Eastern (EEE) and Venezuelan (VEE) equine encephalitis; Semliki Forest (SFV) and Sindbis Viruses (SIN).

5. The avian cell of claim 1, wherein
(i) the alphavirus RNA contains a heterologous target gene to be expressed in a cell; and/or
(ii) the avian cell proliferates in medium free of animal-derived components; and/or
(iii) the avian cell releases replicon particles into the culture supernatant or does not release replicon particles into the culture supernatant; and/or
(iv) the avian cell does not release particle-associated reverse transcriptase activity.

6. The avian cell of claim 1 wherein the permanent host cell is
(i) derived from embryonic or hatched chicken, duck, goose, quail or the like; and/or
(ii) derived from duck somites or neuronal tissue; and/or
(iii) is immortalized with a combination of viral and/or cellular genes (gene(s)), at least one first gene affecting the function of the retinoblastoma protein by mediating disruption of complexes between retinoblastoma proteins and E2F transcription factors and at least one second gene affecting the p53 protein or a family member thereof.

7. The avian cell of claim 1 wherein the permanent host cell is deposited with the DSMZ under accession number DSM ACC2749.

8. The avian cell of claim 1, wherein the permanent host cell is derived from a permanent cell line with a stable karyotype for at least 50 passages, does not release particle associated reverse transcriptase and grows in medium free of animal components

9. A method for preparing an avian cell as defined in claim 1 which comprises stably maintaining at least one DNA sequence for the alphavirus polypeptide in the permanent avian cell wherein the alphavirus polypeptide is
(i) derived from Eastern (EEE) and Venezuelan (VEE) equine encephalitis; Semliki Forest (SFV) and Sindbis Viruses (SIN); and/or
(ii) the structural protein from an alphavirus with non-mammalian animals as reservoir host or is the structural protein from an alphavirus with mammals as reservoir host species; and/or
(iii) the non-structural protein from an alphavirus with non-mammalian animals as reservoir host or is the non-structural protein from an alphavirus with mammals as reservoir host species.

10. An alphavirus replicon particle produced by the avian cell of claim 1.

11. A method to produce alphavirus replicon particles in an avian cell as defined in claim 1 which comprises contacting a cell of claim 1 with corresponding alphavirus replicon RNA, wherein “contacting”
(i) occurs by means of transduction with replicon particles or viral carrier at an MOI below 1; and/or
(ii) occurs by means of transfection of replicon RNA or by transfection of DNA driving expression of the replicon RNA.


SEQUENCE LISTING (21 sequences)

SEQ ID NO: 1 - skipped

SEQ ID NO: 2 - DNA, 3069 bp, Artificial
  E1B Cassette
caaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggtgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctcaatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaataccgagcgaccctgcagcgacccgcttaacagcgtcaacagcgtgccgcagatcgctaccggactcagatcccctacctgcgcctctcttccccagacctgcgcgctactgcggctcgggcggtcgctcgcctggctctgctccatttgactgtctgtgtgcagtcgcagaacttcgaagagggttttgcgctccatccgtggcgtttcgcttttgttcggttttgttgtttatttcattttttttttccggagagaggcgaggcggtggtccacacccgcccgaggaggaaggatctcgagcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggataacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggca

SEQ ID NO: 3 - protein, 264 aa, Artificial
  SIN Capsid
MNRGFFNMLGRRPFPAPTAMWRPRRRRQAAPMPARNGLASQIQQLTTAVSALVIGQATRPQPPRPRPPPRQKKQAPKQPPKPKKPKTQEKKKKQPAKPKPGKRQRMALKLEADRLFDVKNEDGDVIGHALAMEGKVMKPLHVKGTIDHPVLSKLKFTKSSAYDMEFAQLPVNMRSEAFTYTSEHPEGFYNWHHGAVQYSGGRFTIPRGVGGRGDSGRPIMDNSGRVVAIVLGGADEGTRTALSVVTWNSKGKTIKTTPEGTEEW

SEQ ID NO: 4 - protein, 981 aa, Artificial
  SIN Glycoprotein
MSAAPLVTAMCLLGNVSFPCDRPPTCYTREPSRALDILEENVNHEAYDTLLNAILRCGSSGRSKRSVTDDFTLTSPYLGTCSYCHHTEPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKTTAGYITMHRPGPHAYTSYLEESSGKVYAKPPSGKNITYECKCGDYKTRTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAVHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQAASGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 5 - DNA, 2717 bp, Artificial
  EF2 and VEE UTR up to ATG
cagcggagcttctctgcagaggtgaatttttttattggtccccaccctaaaagacccagcgtatatttattattgtacttattacagctgacctggttgtggctgcttgtggtactccaggattctgctcttcatggatgtggggatgacagtgggagagatccttccctgccaacacctggctccatccctgcagttatggggttgaaaattcccctttttcccttaagctctccccctagttctttaaattacatgatgttagaatttttcaataagcaacccatgcacttgagaagaaattcaacccctcttttattaagttccgggatacctgtgaacagccccccctcctttttttttttttttttttttgagacagagtcttactctcagtcgcacaggctggagtgcatgcagtgccgtgatctcggctcactgcaacctctgcctctcgggttcaagcggttctcctgcctcagcctccaaagtagctgggatttcaggcgttcactaccacgccgggctaatttttttatttttagtaaagacggggtttctccatattgggtaggctggtctcgaactcctgacttcaggtgatttgcctgcctcggcctcccaaagtgctgggattacaggcgtgatccaccgcaagcagcctaacccctctttaatttttaaaatttttaaaaatttttttgagacggaatctcgctctgtcgccaggctggagagcagtggcgcgatctcggctaactgcaacctccgcctctcgcctgacacctcttttaatgaaaacctttccattacatctctgaaatgctcccactttgcacggaaaaaacggctgcgaagcccccaataggaaacgcaacacgtagcaggtgctcagtgcatttaaaaattttgaagaattttattattaataatgcgaaactgactacccctctgctaaaaactcatgccccaaatggcgcatgcgcttgacttgcaacttaactaaacgcgtcacaggtacaggtgtaggtggttagggaccccgggcgctcccatctgctccctgccgcgccctctgcctttcagagcctcaatcctttcggaagatacttttcagttttccactgggttaaatggtgataggaagagcaatcatccgttaaatgaaacccaaggttgagacagcgctttgtaaagcaaagtgcccgaagcattcgtgagggggaggagtgtacaggtttattttagagtccaggctggaatttgcattgctttttgtggatccacacctccccctctcccagcccaggaaaagcactgcagtggctctcctgtttcacaaagtgagtttaaggaaagatgtaccgcagggggttcatttgcactgtgagaacatcattcatgcaagaaatgctgagtacctactatgtgctaaacagcgggttgggcttttcataagggatagagttacacaattgaatgccaattcatgagatatgaaatacattcaactgtttcttagggtcagattaggacacaaatgctaaacagtcttcctgcgcctccccctggagaggaaatgaaatagaaaatgaaattgtgccagtaaaccgcttagccgggtgcctgggacatggcaaaccagtcctgaaataaataactgttttattaatagcaatcttagctaattaaaatagatagcgtttattgagcgttgggtatcaggcacggtcctaattcttttagatgtctttagttcgtttcactctcccccaaacaatagggtggtattgatcacctccgagcaggtgaaattgaggcacagagaaatcctagtagctggtagaagaacacgcagtgtggtcaagctagcaaggtgtttggtccactgctatatctacaaaacccctaacaatgcctggtgtatagatgctcagtatgcatttgtgggatcagtgattccgatgcctgcttcttataaagtttttatttagaaataattacaggtaaggagttgcaaaaacagtatagtgggatcgagtgtcctttttccctcggcttctcccaggggtatcgtcttacgtaacaatgtccaaacagggaaattgacttgggtataatccacagactctattcaccttgtaaattggttttaatagaagtatcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtagggatactggacaacttgtaagctaatatcgttgctatggttctcgttctcagctaaaacggcgctctttactttgtgcacctgaacactgcacaccgagggcgaccaccgcccccgagatgcccagcttctattctagagcgccgcgccggcgccgaatgggttaacgggcggggggacacgcctccgtgcgcttgcgcggcgtcccttcgccccgccttcgcagcgcagtcacatgacccgcccaaccggcgtccgccttatataagcagagctcgtttagtgaaccgtatgggcggcgcatgagagaagcccagaccaattacctacccaaaatg

SEQ ID NO: 6 - DNA, 580 bp, Artificial
  mCMV and VEE UTR up to ATG
aattcgtttatcggccaccgaggccaagatctggccgccatatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttataaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtatataagcagagctcgtttagtgaaccgtatgggcggcgcatgagagaagcccagaccaattacctacccaaaatg

SEQ ID NO: 7 - DNA, 251 bp, Artificial
  VEE 3′ UTR and HDV
atacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagagccactttt

SEQ ID NO: 8 - DNA, 24 bp, Artificial
  VEE sg
ctctctacggctaacctgaatgga

SEQ ID NO: 9 - DNA, 7521 bp, Artificial
  VEE nsP and SIN Packaging Signal
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagggggatcacgggagaaaccgtgggatacgcggttacacacaatagcgagggcttcttgctatgcaaagttactgacacagtaaaaggagaacgggtatcgttccctgtgtgcacgtacatcccggccaccataccctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaa

SEQ ID NO: 10 - DNA, 20 bp, Artificial
  Primer
cagcgtaacggttagcatag

SEQ ID NO: 11 - DNA, 20 bp, Artificial
  Primer
cgtttggtcgctcttatagg

SEQ ID NO: 12 - DNA, 20 bp, Artificial
  Primer
cattggacaggcaactagac

SEQ ID NO: 13 - DNA, 20 bp, Artificial
  Primer
ctttcacgtgcagaggtttc

SEQ ID NO: 14 - DNA, 20 bp, Artificial
  Primer
cttccatgaattcgcctacg

SEQ ID NO: 15 - DNA, 20 bp, Artificial
  Primer
tctgagagtacctgcatgac

SEQ ID NO: 16 - DNA, 18 bp, Artificial
  Primer
tggtgaccccgacgtgat

SEQ ID NO: 17 - DNA, 23 bp, Artificial
  Primer
cctatttccttcttagaaggaga

SEQ ID NO: 18 - DNA, 22 bp, Artificial
  Primer
cgatctccttcccggaaggagt

SEQ ID NO: 19 - DNA, 21 bp, Artificial
  Primer
gcctttgagagttactctttg

SEQ ID NO: 20 - DNA, 23 bp, Artificial
  Primer
aaacactgtacggcacccgcatt

SEQ ID NO: 21 - DNA, 21 bp, Artificial
  Primer
gcctttgagagttactctttg